(19) United States
(12) Patent Application Publication
(10) Pub. No.: US 2004/0006813 A1
(43) Pub. Date: Jan. 15, 2004

(54) SWIMMING GOGGLES WITH IMPROVED ADJUSTABILITY

(76) Inventor: Herman Chiang, Chung-Ho City (TW)

Correspondence Address: Keith Kline, PRO-TECHTOR INTERNATIONAL SERVICES, 20775 Norada Court, Saratoga, CA, 95070-3018, US

(21) Appl. No.: 10/194,092

(22) Filed: Jul. 11, 2002

(51) Int. Cl. A61F 9/02

(52) U.S. Cl. 2/428

(57) ABSTRACT

A pair of swimming goggles for swimming pool purposes comprising: a left and a right lens frame each of having a lens unit, a nose bridge connecting an inner sides of the left and right lens frame, and a strap head, wherein the nose bridge is made of flexible and rigid material, including a body portion for touching wearer's nose, a guide portion with a first bar and a second bar which are respectively extended from on the opposite sides of said body portion, each of said first bar and said second bar have a parallel section for providing the lens frames to move thereon, and a connection portion for connecting the strap head, such the swimming goggles can effectively guard against interference each other during the adjusting and can respectively conform with a wearer's eyehole contour, so as to provide more comfortable fitting and prevent the seepage of water when the swimming goggles are in use.

### FIELD OF THE INVENTION

The present invention relates generally to swimming goggles, in particular to a pair of swimming goggles that is improved about its nose bridge which can be conform with wearer's nose by configuration, and which can effectively guard against interference each other during adjusting, such that it can provide wearer have more comfortable and best prevent the seepage of water when the swimming goggles is in use.

### BACKGROUND OF THE INVENTION

Swimming goggles have been known for many years and its nose bridge have two common types by function, comprising a fixing nose bridge type which is generally formed integrally with lens frames, an adjustable nose bridge type which is connected to lens frames and can be adjusted for difference spacing, and it was well known in the prior art, for example, U.S. Pat. Nos. 5,502,844, 5,857,221, 5,950,248, 6,119,277, 6,119,279 etc. However, all the adjustable nose ridge type have main drawback that wearer have to take off the swimming goggles from her/his head before adjusting, and both lens frames of the swimming goggles shall be effected each other and be moved toward left or right during wearer adjusting the lens frames match her or his eyehole, so it is impossible that no matter how she or he has done their best in that way, the lens frames can not correctly and securely match wearer's each eyehole. So that the swimming goggles with adjustable nose bridge type of the above prior arts not only is inconvenient but also is uncomfortable in use. Moreover, it is uncomfortable that the nose bridge contact with wearer's nose after user wore the swimming goggles, because wearer's nose is sloped and the nose bridge is planar so that wearer's nose have heavy press in contact.

### OBJECTS OF THE INVENTION

An object of the present invention is to provide a swimming goggles with a adjustable nose bridge that can overcome the drawbacks of the aforementioned prior art, the nose bridge is sloped configuration which can be matched wearer's shape of the nose, and it can be connected lens frames of the swimming goggles to provide each lens frame can be adjusted on the nose bridge, and which can be joined a head strap together, so that each lens frame do not effect each other during adjusting, and can be securely matched with the periphery of wearer's eyeholes respectively and get more comfortable and prevent effectively the seepage of water when the swimming goggles is in use.

Another object of the present invention is to keep each lens frame parallel moving with wearer's eyeball during adjusting, causing a lens of the each lens frame is also parallel with wearer's eyeball, and to allow good peripheral vision without distorting the swimmer's view.

According to one feature of the present invention, a nose bridge is made of flexible and rigid material, which includes a body portion for touching a wearer's nose, a guide portion with a first bar and a second bar which are respectively extended from on the opposite sides of said body portion, each of said first bar and said second bar have a parallel section, and a connection portion for connecting a strap head.

According to the above mention feature, the body portion have outer surface and inner surface for touching wearer's nose, the inner surface has a upper edge and a lower edge is located in different orientation, so as to conformed with the slope of wearer's nose after wear.

According to another feature of the present invention, a connection base is protruded on an upper and a lower of periphery of each lens frame for holding during each lens frame is adjusted. The connection base has a receiving hole for providing the first bar and the second bar passing through respectively, and the shape and size of the receiving holes are the same with the longitudinal section of the first bar and the second bar in order to move secure each lens frames is moved along the first bar and the second bar, thus causing a lens unit of each lens frame to be parallel with wearer's eyeball.

According to more featured of the present invention, a fastener mechanism includes an assemble section is coupled with said connection portion and a strap linking section is connected to a head strap.

### BRIEF DESCRIPTION OF THE DRAWINGS

Other features and advantages of the present invention will become apparent in the following detailed description of the preferred embodiments with reference to the accompanying drawings of which:

FIG. 1 is a perspective view of the disassembled parts of the swimming goggles of the present invention;

FIG. 2 is a perspective view of the part assembled of the swimming goggles in FIG. 1;

FIG. 3 is a top view of the swimming goggles in FIG. 2;

FIG. 4 is a perspective view of the nose pad of the present invention;

FIG. 5 is a front view of the swimming goggles in FIG. 1;

FIG. 6 is a cross-section view of the swimming goggles along line 6-6 of FIG. 5;

FIG. 7 is a cross-section view of the swimming goggles along line 7-7 of FIG. 5.

### DETAILED DESCRIPTIION OF THE PREFERRED EMBODIMENT

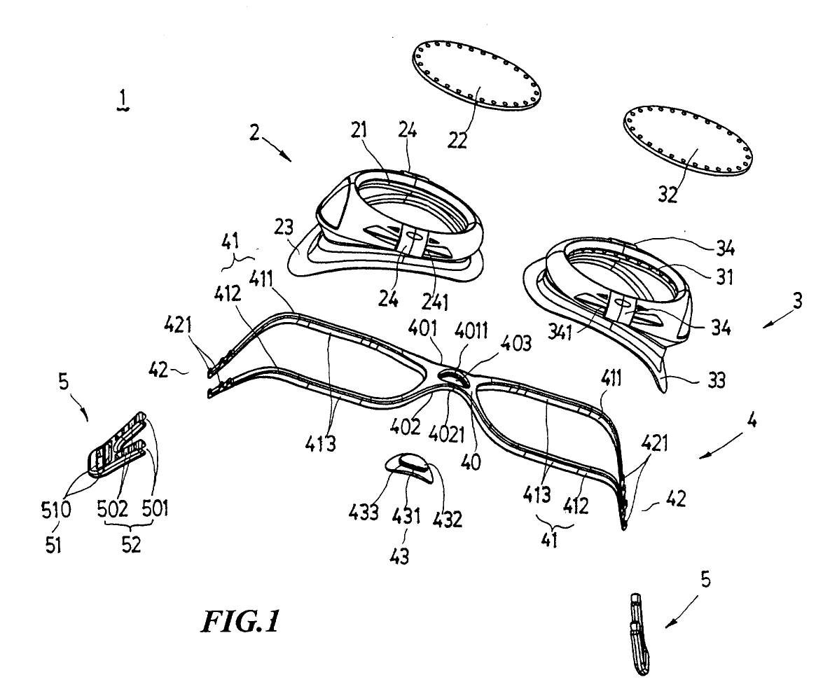

Referring to FIG. 1, the preferred embodiment of a swimming goggles 1 according to the present invention is show to comprise a right lens frame 2, a left lens frame 3, a nose bridge 4 and a fastener mechanism 5 and a strap head (not shown), wherein each of the lens frames 2,3 has a lens retaining spaces 21,31 for molding insert a lens units 22,32 thereon. The right lens frame 2 and the left lens frame 3 respectively has a gaskets 23,33 each of which is formed integrally with far away from the side of each of the lens units 22,32 for touching the periphery of a wearer's eyehole. The upper and lower of periphery of the lens frames 2,3 respectively have a connection bases 24,34 which are protruded thereon for holding when each lens frame 2,3 is adjusting. A receiving holes 241,341 are respectively disposed on the connection bases 24,34.

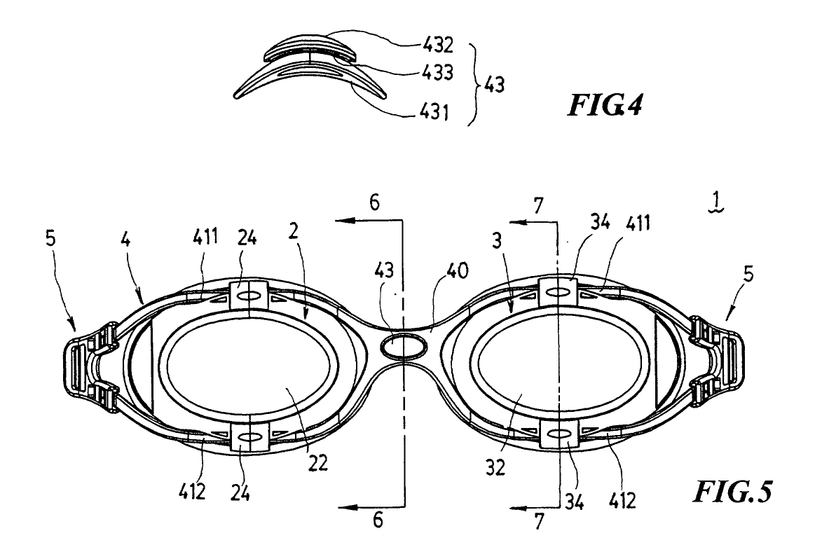

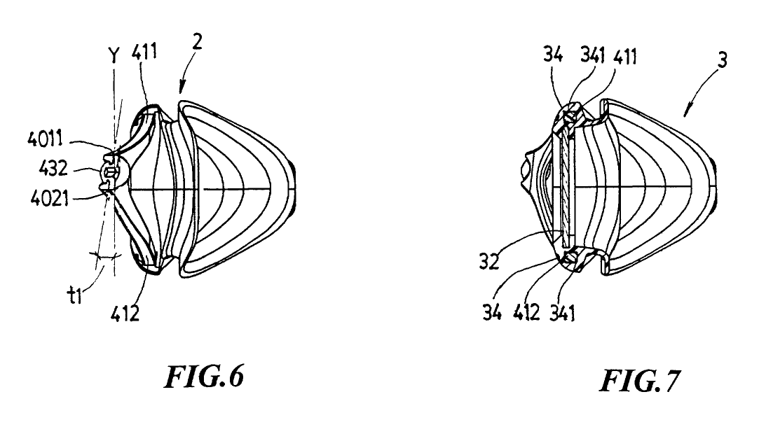

Referred to FIGS. 1, 5 and 6, the nose bridge 4 is made of flexible and rigid material, which includes a body portion 40 has an outer surface and inner surface for touching wearer's nose, the inner surface have a upper edge 401 and a lower edge 402, a line continue alone the highest 4011 of the upper edge 401 and the lowest 4021 of the lower edge 402 forming an angle t1 with the Y axial(as shown in FIG. 6), it should be understand the inner surface of the upper edge 401 and the lower edge 402 are located in different orientation, so as to conformed with the slope of wearer's nose after wear. A hole 403 is mounted on the body portion 40 for assembly a nose pad 43, referred to FIG. 4 the nose pad 43 have a conformable portion 431 for touching nose and a joining portion 432 is integrally formed with the conformable portion 431, which has a neck 433 which is engaged to the hole 403 of the body portion 40, and the conformable portion 431 is generally a arc shape for match with wearer's nose. A guide portion 41 has a first bar 411 and a second bar 412 which are respectively extended from the upper edge 401 and the lower edge 402 of the body portion 40, and cooperate with each other to form a fork shape, it will be more conformable with wearer's nose and more comfortable through the body portion 40, the first bar 411 and the second bar 412 in wear. Moreover, each of said first bar 411 and said second bar 412 has a parallel section 413 for providing each lens frame moving thereon. In FIG. 7, the shape and size of the receiving holes 241,341 are the same with the longitudinal section of the first bar 411 and the second bar 412 in order to move secure when each lens frames 2,3 is moved along the first bar 411 and the second bar 412, thus causing a lens unit 22,23 to be parallel with wearer's eyeball. A connection portion 42 is extended from the end of the parallel section 413 of the first bar 41 land the second bar 412 and is bended near the lens frames 2,3, a several stoppers 421 are located on the connection portion 42.

A fastener mechanism 5 is generally a board includes an assembled section 50 is coupled with said connection portion 42 and a strap linking section 51 is connected to said head strap, wherein the assembled section 50 includes two concave housings 501 which are mounted on the board for accommodation the first bar 411 and the second bar 412 respectively, and a several openings 502 which are respectively disposed on the concave housings 501 for engaging the stoppers 421. The strap linking section 51 includes two assembled holes 510 which are mounted on the board for positioning the strap head.

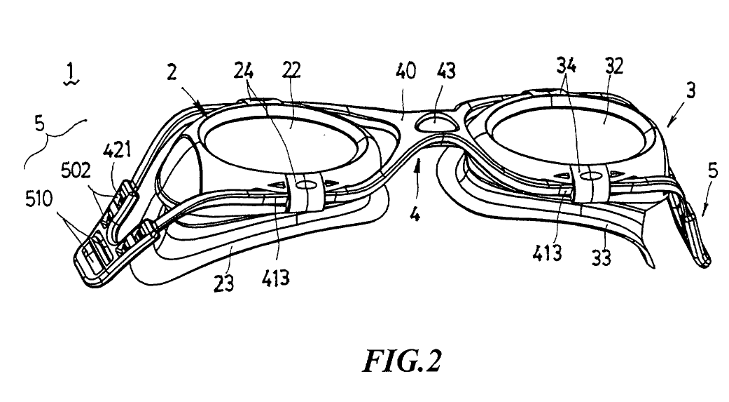

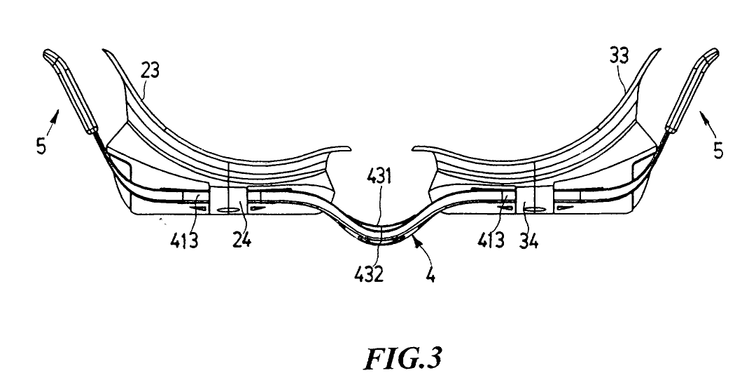

Referring to FIG. 2 and 3, during assembly, the lens units 22,32 is inserted into the lens retaining spaces 21,31 of the lens frames 2,3. The lens frames 2,3 are joined together by the first bar 411 and the second bar 412 passing through the receiving holes 241,341 and then the stoppers 421 of each connection portion 42 of the first bar 41 land the second bar 412 are engaged to the openings 502 of each concave housings 501 of the fastener mechanism 5, so that the lens frames 2,3 should be securely assembled into one unit. After assembly, the connection bases 24,34 are located on the parallel section 413 of the first bar 41 land the second bar 412, and again referred to FIG. 7, it is obvious that the shape and size of the receiving holes 241,341 are the same with the longitudinal section of the first bar 411 and the second bar 412 in order to move secure when each lens frames 2,3 is moved along the first bar 411 and the second bar 412, thus causing a lens unit 22,23 to be parallel with wearer's eyeball., and to allow good peripheral vision without distorting the swimmer's view. Further moreover, wearer do not take off swimming goggles during adjusting because each lens frame 2,3 can be respectively moved on the parallel section 413 of each of the first bar 411 and the second bar 412, and do not effect each other during adjusting, and can be securely matched with the periphery of wearer's eyeholes respectively and get more comfortable and prevent effectively the seepage of water when the swimming goggles is in use.

While the present invention has been described in connection with what is considered the most practical and preferred embodiment, it is understood that this invention is not limited to the disclosed embodiments but is intended to cover various arrangements included within the spirit and scope of the broadest interpretation so as to encompass all such modifications and equivalent arrangements, for example the connection bases 24,34 of the lens frames 2,3 comprise accommodating hole with clasp opening for securing the first bar 411 and the second bar 412 instead of the receiving holes 241,341, or deleting the connection portion 42 to connect the strap head on the outer side of each lens frame, or the nose pad is made of foam instead of plastics and be directly fixed on the body portion by agglutinant etc.,.

What is claimed is:

1. Swimming goggles with improved adjustability comprising:
left and right lens frame, each of having an inner periphery that defines a lens retaining space for receiving a lens unit, a upper and lower of periphery of the left and the right lens frame having at least a connection base with a receiving hole thereon,
a nose bridge connecting an inner sides of the left and right lens frame,
a head strap, wherein;
the nose bridge is made of flexible and rigid material including:
a body portion for touching a wearer's nose,
a guide portion having a first bar and a second bar being respectively extended from an opposite sides of the body portion for passing through the receiving hole of each the connection base, and
a connection portion extending from an end of the first bar and the second bar respectively,
a fastener mechanism for connecting the head strap having an assembled section be able to coupling with the connection portion, and a strap linking section be able to connect of the head strap.

2. The swimming goggles with improved adjustability as claimed in claim 1, wherein each of the first bar and the second bar has a parallel section for providing the left and right lens frame to move thereon.

3. The swimming goggles with improved adjustability as claimed in claim 1, wherein the connection base is protruded on an upper and a lower of periphery of the left and right lens frame for holding during each lens frame is adjusted.

4. The swimming goggles with improved adjustability as claimed in claim 1, wherein the connection portion is extended from the end of the parallel section of the first bar and the second bar and is bended near the lens frame, a several stoppers are located on the connection portion.

5. The swimming goggles with improved adjustability as claimed in claim 1, further comprising a nose pad have a conformable portion for touching nose and a joining portion is integrally formed with the conformable portion, which has a neck which is engaged to a hole of the body portion.

6. The swimming goggles with improved adjustability as claimed in claim 1, wherein the body portion have an outer surface and inner surface for touching wearer's nose, the inner surface has a upper edge and a lower edge which is located in different orientation, so as to conformed with the slope of wearer's nose after wear.

7. The swimming goggles with improved adjustability as claimed in claim 6, wherein the first bar and the second bar are respectively extended from the upper edge and the lower edge of the body portion and cooperate with each other to form a fork shape.

8. The swimming goggles with improved adjustability as claimed in claim 7, wherein the fastener mechanism is generally a board, the assembled section includes two concave housings which are mounted on the board for accommodation the first bar and the second bar respectively, a several openings which are respectively disposed on the concave housings for engaging the stoppers, the strap linking section includes two assembled holes which are mounted on the board for positioning the strap head.

9. The swimming goggles with improved adjustability as claimed in claim 1, wherein the shape and size of the receiving holes are the same with the longitudinal section of the first bar and the second bar in order to move secure when each lens frames is moved along the first bar and the second bar, thus causing each lens unit to be parallel with wearer's eyeball.

10. Swimming goggles with improved adjustability comprising:
left and right lens frame, each of having an inner periphery that defines a lens retaining space for receiving a lens unit, a upper and lower of periphery of the left and right lens frame having at least a connection base with a receiving hole thereon, and a strap head connecting an outer side of the left and right lens frame,
a nose bridge connecting the left and right lens frame, wherein;
the nose bridge is made of flexible and rigid material including:
a body portion has an outer surface and inner surface for touching a wearer's nose,
a guide portion having a first bar and a second bar with a parallel section being respectively extended from an opposite sides of the body portion for passing through the receiving hole of each the connection base.

11. The swimming goggles with improved adjustability as claimed in claim 10, the connection base is respectively protruded on an upper and a lower of periphery of the left and right lens frame for holding during each lens frame is adjusted.

12. The swimming goggles with improved adjustability as claimed in claim 11, the shape and size of the receiving holes are the same with the longitudinal section of the first bar and the second bar in order to move secure when the first bar and the second bar are moved around the receiving holes, thus causing a lens unit to be parallel with wearer's eyeball.

13. Swimming goggles with improved adjustability comprising:
left and right lens frame, each of having an inner periphery that defines a lens retaining space for receiving a lens unit, a upper and lower of periphery of the left and right lens frame having at least a connection base with a receiving hole thereon, and a strap head be located on an outer side of the left and right lens frame,
a nose bridge connecting the left and right lens frame, wherein the nose bridge including:
a body portion with a nose pad on an inner thereof which comprises a conformable portion for touching nose and a joining portion for engaging the body portion,
a guide portion with a first bar and a second bar being respectively extended from on the opposite sides of the body portion for passing through the receiving hole of the connection base.

14. The swimming goggles with improved adjustability as claimed in claim 13, wherein the joining portion is integrally formed with the conformable portion having a neck is engaged to a hole of the body portion.

15. The swimming goggles with improved adjustability as claimed in claim 14, wherein the conformable portion is generally a arc shape for matching with wearer's nose.
